(19) United States
(12) Patent Application Publication
(10) Pub. No.: US 2026/0108394 A1
(43) Pub. Date: Apr. 23, 2026

(54) EYE MASK WITH ADJUSTABLE EYECUP SPACING

(71) Applicant: Zhenyu Liu, Shenzhen (CN)

(72) Inventor: Zhenyu Liu, Shenzhen (CN)

(21) Appl. No.: 19/424,628

(22) Filed: Dec. 18, 2025

(51) Int. Cl. A61F 9/04

(52) U.S. Cl. CPC A61F 9/045

(57) ABSTRACT

An eye mask with adjustable eyecup spacing is provided, including a first eyecup body, a second eyecup body, an eyecup spacing adjustment strap connected between the first eyecup body and the second eyecup body at their sides close to each other, and a head binding strap connected between the first eyecup body and the second eyecup body at their sides far away from each other, wherein the eyecup spacing adjustment strap is configured to enable the first eyecup body and the second eyecup body to move closer to or further away from each other. The eye mask is simple in structure, easy in operation, and adjustable in eyecup spacing, with good applicability.

### TECHNICAL FIELD

The present disclosure relates to the technical field of eye care devices, more particularly to an eye mask with adjustable eyecup spacing.

### BACKGROUND

With the development of science and technology, electronic devices are becoming increasingly popular, the pace of work and life are getting faster and faster, and people's eye strain has significantly increased, thus problems such as eye fatigue, dryness, and soreness have become common health problems. One of the key reasons for these problems is excessive evaporation of water on the ocular surface. Factors such as air conditioning environment, dry climate, etc. can further accelerate the loss of water from the ocular surface, resulting in eye dryness, sensation of foreign bodies and other discomforts. Conventional eye-shut rest or sleep is difficult to recover the fatigue of eyes, and the loss of water can continue during sleep, leading to a vicious cycle of fatigue-dryness-more fatigue.

To relieve the above problems, eye masks are widely used in eye care to maintain eye moisture when users are taking a rest, thus fully relieving the fatigue feel of eyes. However, most existing eye masks are just designed to be suitable for users with different head circumferences, without taking into account different interpupillary distances. They are unable to meet simultaneously the requirements of users with different interpupillary distances and are incapable of fully fitting the eye sockets of users, leading to a poor care effect. Therefore, it is necessary to provide an improved technical scheme.

### SUMMARY

In view of the above, the present disclosure aims to address at least one of the technical problems in existing technologies. To achieve this aim, the present disclosure provides an eye mask with adjustable eyecup spacing, which can achieve eyecup spacing adjustment by arranging a first eyecup body and a second eyecup body to move closer to or further away from each other, thereby meeting the requirements of users with different interpupillary distances, including position correspondence and fitness during the usage of the eye mask.

To achieve this aim, in a first aspect, the present disclosure provides an eye mask with adjustable eyecup spacing, including a first eyecup body, a second eyecup body, an eyecup spacing adjustment strap connected between the first eyecup body and the second eyecup body at their sides close to each other, and a head binding strap connected between the first eyecup body and the second eyecup body at their sides far away from each other, wherein the eyecup spacing adjustment strap is configured to enable the first eyecup body and the second eyecup body to move closer to or further away from each other.

Preferably, the eyecup spacing adjustment strap is arranged in an elastic strap structure; and/or
at least one of the first eyecup body and the second eyecup body is moveably connected to the eyecup spacing adjustment strap in a manner of moving closer to or further away from the other one.

Preferably, the eyecup spacing adjustment strap and the head binding strap are arranged as an integrated elastic extension strap, the elastic extension strap passing through the first eyecup body and the second eyecup body respectively.

Preferably, the first eyecup body includes a first base and a first eye contour part extended toward one side from the first base, and the second eyecup body includes a second base and a second eye contour part extended toward one side from the second base, the first eye contour part and the second eye contour part being configured to conform to a pair of eye contours, and the elastic extension strap passing through the first base and the second base respectively.

Preferably, the first base is formed with a through first extension slot, and the second base is formed with a through second extension slot, the elastic extension strap being moveably connected to the first extension slot and the second extension slot respectively.

Preferably, the first base is further formed with a first disengagement slot communicated with the first extension slot, and the second base is further formed with a second disengagement slot communicated with the second extension slot, the elastic extension strap being capable of disengaging from or connecting to the first eyecup body via the first disengagement slot, and disengaging from or connecting to the second eyecup body via the second disengagement slot; and/or,
the first extension slot is arranged in a flat slot structure parallel to the first eye contour part, and the second extension slot is arranged in a flat slot structure parallel to the second eye contour part, the elastic extension strap being arranged in a corresponding flat strap structure.

Preferably, the first eye contour part and the second eye contour part are provided with a first limit tab and a second limit tab respectively at their sides far away from each other, the elastic extension strap passing through the first limit tab and the second limit tab respectively.

Preferably, the elastic extension strap is arranged in a ring-shaped structure, the elastic extension strap being provided with a connection piece at a middle part of the head binding strap.

Preferably, the head binding strap is arranged in an elastic strap structure; and/or, at least one of the first eyecup body and the second eyecup body is moveably connected to the head binding strap in a manner of moving closer to or further away from the other one.

Preferably, the first eyecup body and the second eyecup body are made of a flexible silicone material.

The present disclosure provides an eye mask with adjustable eyecup spacing, including a first eyecup body and a second eyecup body, which are configured to conform to a user's eyes. An eyecup spacing adjustment strap is employed to connect the first eyecup body and the second eyecup body and to enable them to move closer to or further away from each other to adjust the relative distance therebetween, thereby meeting the requirements of users with different interpupillary distances. The first eyecup body and the second eyecup body can be adjusted to correspond to and closely fit around the user's eyes. The eye mask with adjustable eyecup spacing is simple in structure, easy in operation, and high in scalability, providing good experiences to users.

### BRIEF DESCRIPTION OF THE DRAWINGS

FIG. 1 is a perspective view of an eye mask with adjustable eyecup spacing according to the present disclosure.

FIG. 2 is a perspective view of the eye mask with adjustable eyecup spacing shown in FIG. 1 from another angle of view.

FIG. 3 is a front view of the eye mask with adjustable eyecup spacing shown in FIG. 1.

FIG. 4 is a top view of the eye mask with adjustable eyecup spacing shown in FIG. 1.

FIG. 5 is a right view of the eye mask with adjustable eyecup spacing shown in FIG. 1.

FIG. 6 is an exploded view of the eye mask with adjustable eyecup spacing shown in FIG. 1.

### DETAILED DESCRIPTION OF THE EMBODIMENTS

The embodiments of the present disclosure are described below in detail, examples of which are shown in the accompanying drawings on which the same or similar designators are used for representing the same or similar elements or elements having the same or similar functions. The following embodiments described in combination with the drawings are exemplary and intended to explain the present disclosure, but they should not be construed as a limitation on the present disclosure.

The following content provides many different embodiments or examples for implementing different structures of the present disclosure. In order to simplify the present disclosure, the components and settings of specific examples will be described below. Of course, they are examples merely and are not intended to limit the present disclosure. In addition, the present disclosure can repeat reference numbers and/or letters in different examples. This repetition is for the purpose of simplification and clarity, not indicating the relationship between the various embodiments and/or settings discussed herein. In addition, the present disclosure provides examples of various specific processes and materials, but those ordinary skill in the art should recognize the applicability of other processes and/or the use of other materials.

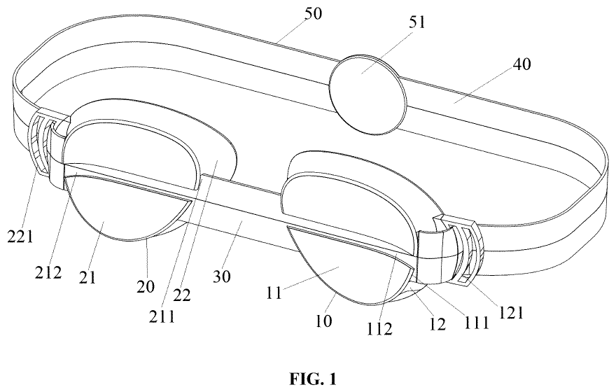

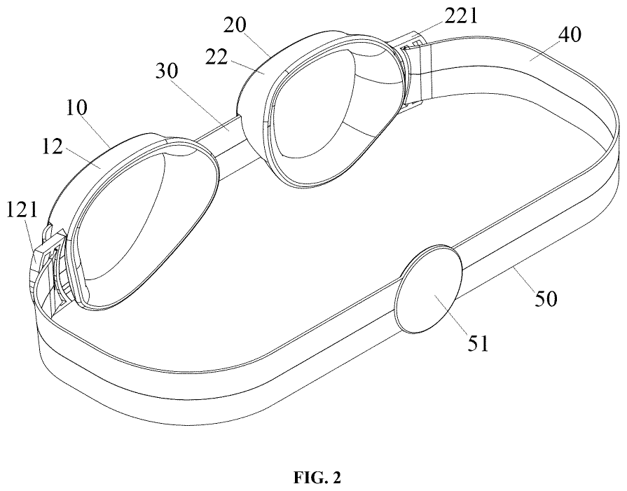

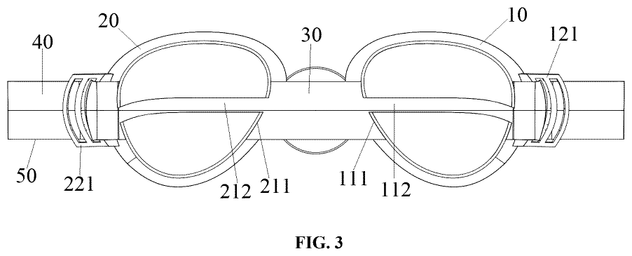

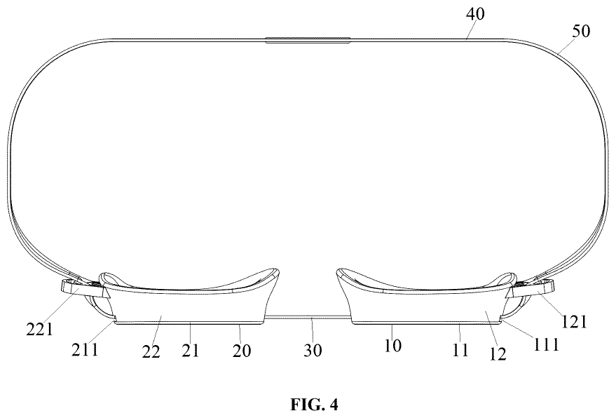

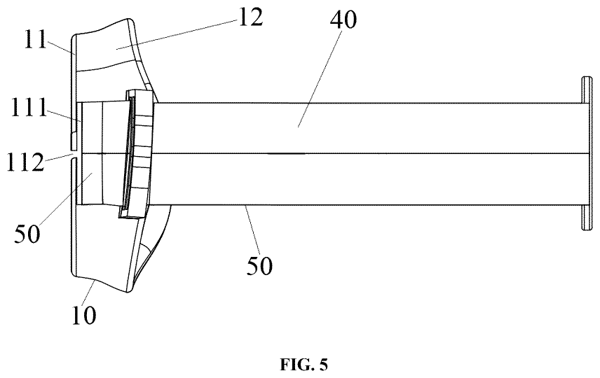

Referring to FIG. 1 to FIG. 5, it is an object of the present disclosure to provide an eye mask with adjustable eyecup spacing, which is used for eye care, for example, eye moisturizing care during sleep. The eye mask with adjustable eyecup spacing includes a first eyecup body 10, a second eyecup body 20, an eyecup spacing adjustment strap 30 connected between the first eyecup body 10 and the second eyecup body 20 at their sides close to each other, and a head binding strap 40 connected between the first eyecup body 10 and the second eyecup body 20 at their sides far away from each other, wherein the eyecup spacing adjustment strap 30 is configured to enable the first eyecup body 10 and the second eyecup body 20 to move closer to or further away from each other.

Specifically, the first eyecup body 10 and the second eyecup body 20 are adapted to fit around a user's eyes, to realize moisturizing care. The eyecup spacing adjustment strap 30 is configured to achieve adjustment of a relative distance between the first eyecup body 10 and the second eyecup body 20, to meet the requirements of users with different interpupillary distances.

Further, the eyecup spacing adjustment strap 30 is arranged in an elastic strap structure. It is understandable that the elastic strap structure can be used to achieve adjustment of the relative distance between the first eyecup body 10 and the second eyecup body 20, hence suiting users with different interpupillary distances.

Further, at least one of the first eyecup body 10 and the second eyecup body 20 is moveably connected to the eyecup spacing adjustment strap 30 in a manner of moving closer to or further away from the other one. It is understandable that the first eyecup body 10 is moveably connected to the eyecup spacing adjustment strap 30, or the second eyecup body 20 is moveably connected to the eyecup spacing adjustment strap 30; alternatively, both of them are moveably connected to the eyecup spacing adjustment strap 30, which can also enable an eyecup spacing adjustment between the first eyecup body 10 and the second eyecup body 20, meeting the requirements of different users. In addition, the moveable connection method and the arrangement of the eyecup spacing adjustment strap 30 in an elastic strap structure can be selected individually or combined; the combination of the elastic strap structure and the moveable connection structure can further improve the convenience and reliability of the eyecup spacing adjustment between the first eyecup body 10 and the second eyecup body 20.

Further, the eyecup spacing adjustment strap 30 and the head binding strap 40 are arranged as an integrated elastic extension strap 50; and the elastic extension strap 50 passes through the first eyecup body 10 and the second eyecup body 20 respectively. It is understandable that, in the present embodiment, the integrated elastic extension strap 50 can realize the head binding function and the eyecup spacing adjustment function and can pass through the first eyecup body 10 and the second eyecup body 20, providing a uniform pressure for the first eyecup body 10 and the second eyecup body 20, thereby the first eyecup body 10 and the second eyecup body 20 are uniformly stressed to closely fit around the eyes of the user.

It is understandable that the eyecup spacing adjustment strap 30 and the head binding strap 40 can be split structures, connected to the first eyecup body 10 and the second eyecup body 20 separately, to realize the eyecup spacing adjustment function and the head binding function.

Further, the first eyecup body 10 includes a first base 11 and a first eye contour part 12 extended toward one side from the first base 11; the second eyecup body 20 includes a second base 21 and a second eye contour part 22 extended toward one side from the second base 21; the first eye contour part 12 and the second eye contour part 22 are configured to conform to a pair of eye contours; and the elastic extension strap 50 passes through the first base 11 and the second base 21 respectively. In the present embodiment, the first eye contour part 12 and the second eye contour part 22 correspond to the eyes of a user respectively; the first base 11 and the second base 21 are arranged roughly in a planar shape; the elastic extension strap 50 passes through the first base 11 and the second base 21, being capable of providing a flat and uniform pressure, enabling the first eye contour part 12 and the second eye contour part 22 to fit the eye contours of the user uniformly to improve the moisturizing effect.

It is understandable that, in other embodiments, the first eyecup body 10 and the second eyecup body 20 are not limited to the above structure. The first base 11 and the second base 21 can also be arranged as a spherical surface, an ellipsoidal surface, etc.

Further, the first base 11 is formed with a through first extension slot 111; and the second base 21 is formed with a through second extension slot 211; and the elastic extension strap 50 is moveably connected to the first extension slot 111 and the second extension slot 211 respectively. In the present embodiment, the first extension slot 111 and the second extension slot 211 are arranged to accommodate the elastic extension strap 50.

It is understandable that, in other embodiments, the first base 11 and the second base 21 can be further provided with a plurality of connection rings, and the elastic extension strap 50 can pass through the connection rings to realize moveable connection; alternatively, the elastic extension strap 50 can be detachably connected to the first base 11 and the second base 21 in a snap-fit manner, where a plurality of snap-fit structures are provided to realize connection at different positions.

Further, the first base 11 is further formed with a first disengagement slot 112 communicated with the first extension slot 111; the second base 21 is further formed with a second disengagement slot 212 communicated with the second extension slot 211; and the elastic extension strap 50 is capable of disengaging from or connecting to the first eyecup body 10 via the first disengagement slot 112, and disengaging from or connecting to the second eyecup body 20 via the second disengagement slot 212.

In the present embodiment, the arrangement of the disengagement slots communicated with the extension slots can facilitate the assembly or separation of the elastic extension strap 50 to or from the first eyecup body 10 or the second eyecup body 20, as well as to realize local structure replacement, for example, the replacement of one eyecup body or the elastic extension strap 50, further improving the scalability. It is understandable that the disengagement slots can be omitted, and the elastic extension strap 50 can be installed by passing through the extension slots.

In addition, the first extension slot 111 is arranged in a flat slot structure parallel to the first eye contour part 12; the second extension slot 211 is arranged in a flat slot structure parallel to the second eye contour part 22; and the elastic extension strap 50 is arranged in a corresponding flat strap structure. In the present embodiment, the arrangement of the elastic extension strap 50 and the extension slot in flat structures can increase the stress area on the eyecup body, further enhancing the uniformity of stress and enabling close fit between the whole eye mask and the user's eye contours.

Further, the first eye contour part 12 and the second eye contour part 22 are provided with a first limit tab 121 and a second limit tab 221 respectively at their sides far away from each other; and the elastic extension strap 50 passes through the first limit tab 121 and the second limit tab 221 respectively. In the present embodiment, the first eyecup body 10 and the second eyecup body 20 are provided with limit tab structures at opposing outer sides thereof, which are configured to limit the elastic extension strap 50, enabling the elastic extension strap 50 to adhere to the first eye contour part 12 and the second eye contour part 22, being conducive to the relative fit to the head. Specifically, the limit tab can also be arranged in an 8-shaped structure, with two neighboring limit holes. In the present embodiment, the integrated elastic extension strap 50 can pass through respective limit holes of the first limit tab 121 and the second limit tab 221 closer to the eye contour parts, while for the split head binding strap 40, its ends can pass through the two limit holes repeatedly to realize connection to the limit tabs, further improving the applicability.

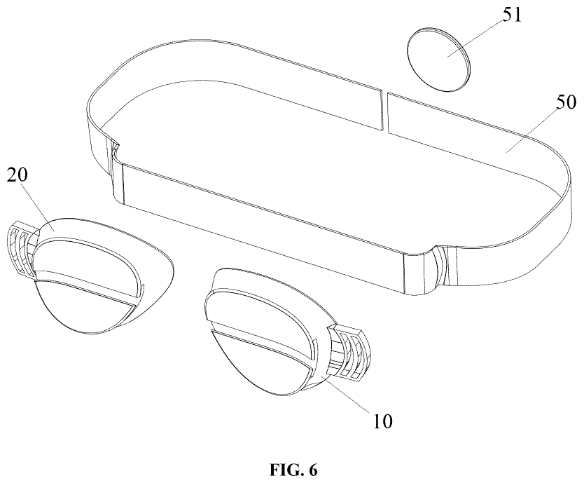

Referring to FIG. 6, further, the elastic extension strap 50 is arranged in a ring-shaped structure; and the elastic extension strap 50 is provided with a connection piece 51 at a middle part of the head binding strap 40. In the present embodiment, the connection piece 51 connects two tail ends of the elastic extension strap 50 to each other. The connection piece 51 can also be used as a positioning reference to facilitate centering of position. Specifically, after the elastic extension strap 5 is assembled with the first eyecup body 10 and the second eyecup body 20, the connection piece 51 can be fixedly connected to the elastic extension strap 50 by hot pressing, or by bonding, snap-fit connection, etc.

Further, the head binding strap 40 is arranged in an elastic strap structure. It is understandable that, in the case that the head binding strap 40 and the eyecup spacing adjustment strap 30 are arranged in split structures, the head binding strap 40 being arranged in an elastic strap structure can meet the usage requirements of users with different head circumferences.

Further, optionally, at least one of the first eyecup body 10 and the second eyecup body 20 is moveably connected to the head binding strap 40 in a manner of moving closer to or further away from the other one. Specifically, one of the first eyecup body 10 and the second eyecup body 20 is moveably connected to the head binding strap 40, or both of them are moveably connected to the head binding strap 40, thereby achieving the effect of shortening or extending the head binding strap 40, further increasing the range of head circumferences and enhancing the applicability.

Further, the first eyecup body 10 and the second eyecup body 20 are made of a flexible silicone material. The flexible silicone material can well fit the user's eye contours, without causing local pressure concentration. It is understandable that the first eyecup body 10 and the second eyecup body 20 can also be made of other flexible materials, such as rubber, organic silicone, etc.; alternatively, just the portion in contact with the user's eye contours is made of a flexible material.

The present disclosure provides an eye mask with adjustable eyecup spacing, including a first eyecup body 10 and a second eyecup body 20, which are configured to conform to a user's eyes. An eyecup spacing adjustment strap 30 is employed to connect the first eyecup body 10 and the second eyecup body 20 and to enable them to move closer to or further away from each other to adjust the relative distance therebetween, thereby meeting the requirements of users with different interpupillary distances. The first eyecup body 10 and the second eyecup body 20 can be adjusted to correspond to and closely fit around the user's eyes. The eye mask with adjustable eyecup spacing is simple in structure, easy in operation, and high in scalability, providing good experiences to users.

In the description of the present specification, terms such as “one embodiment”, “some embodiments”, “specific example”, or “some examples”, etc. mean that the specific features, structures, materials, or features described in combination with the embodiment or example are included in at least one embodiment or example of the present disclosure. In the specification, indicative expressions of the above terms do not necessarily refer to the same embodiments or examples. Moreover, the specific features, structures, materials or features described may be combined in an appropriate manner in any one or more embodiments or examples. In addition, those skilled in the art can combine the different embodiments or examples described in the specification with the features of the different embodiments or examples if no conflict is caused.

Although the embodiments of the present disclosure have been illustrated and described above, it is understandable for the ordinary skill in the art that many modifications, changes, substitutions and transformations may be made to these embodiments without departing from the spirit and principle of the present disclosure. The scope covered by the present disclosure is defined by the claims appended hereinafter and equivalents thereof.

What is claimed is:

1. An eye mask with adjustable eyecup spacing, comprising a first eyecup body, a second eyecup body, an eyecup spacing adjustment strap connected between the first eyecup body and the second eyecup body at their sides close to each other, and a head binding strap connected between the first eyecup body and the second eyecup body at their sides far away from each other, wherein the eyecup spacing adjustment strap is configured to enable the first eyecup body and the second eyecup body to move closer to or further away from each other.

2. The eye mask with adjustable eyecup spacing according to claim 1, wherein the eyecup spacing adjustment strap is arranged in an elastic strap structure; and/or
at least one of the first eyecup body and the second eyecup body is moveably connected to the eyecup spacing adjustment strap in a manner of moving closer to or further away from the other one.

3. The eye mask with adjustable eyecup spacing according to claim 1, wherein the eyecup spacing adjustment strap and the head binding strap are arranged as an integrated elastic extension strap, the elastic extension strap passing through the first eyecup body and the second eyecup body respectively.

4. The eye mask with adjustable eyecup spacing according to claim 3, wherein the first eyecup body comprises a first base and a first eye contour part extended toward one side from the first base, and wherein the second eyecup body comprises a second base and a second eye contour part extended toward one side from the second base, the first eye contour part and the second eye contour part being configured to conform to a pair of eye contours, and the elastic extension strap passing through the first base and the second base respectively.

5. The eye mask with adjustable eyecup spacing according to claim 4, wherein the first base is formed with a through first extension slot, and wherein the second base is formed with a through second extension slot, the elastic extension strap being moveably connected to the first extension slot and the second extension slot respectively.

6. The eye mask with adjustable eyecup spacing according to claim 5, wherein the first base is further formed with a first disengagement slot communicated with the first extension slot, and wherein the second base is further formed with a second disengagement slot communicated with the second extension slot, the elastic extension strap being capable of disengaging from or connecting to the first eyecup body via the first disengagement slot, and disengaging from or connecting to the second eyecup body via the second disengagement slot; and/or
wherein the first extension slot is arranged in a flat slot structure parallel to the first eye contour part, and wherein the second extension slot is arranged in a flat slot structure parallel to the second eye contour part, the elastic extension strap being arranged in a corresponding flat strap structure.

7. The eye mask with adjustable eyecup spacing according to claim 4, wherein the first eye contour part and the second eye contour part are provided with a first limit tab and a second limit tab respectively at their sides far away from each other, the elastic extension strap passing through the first limit tab and the second limit tab respectively.

8. The eye mask with adjustable eyecup spacing according to claim 3, wherein the elastic extension strap is arranged in a ring-shaped structure, the elastic extension strap being provided with a connection piece at a middle part of the head binding strap.

9. The eye mask with adjustable eyecup spacing according to claim 1, wherein the head binding strap is arranged in an elastic strap structure; and/or,
wherein at least one of the first eyecup body and the second eyecup body is moveably connected to the head binding strap in a manner of moving closer to or further away from the other one.

10. The eye mask with adjustable eyecup spacing according to claim 1, wherein the first eyecup body and the second eyecup body are made of a flexible silicone material.
